(19) United States
(12) Patent Application Publication
(10) Pub. No.: US 2024/0066291 A1
(43) Pub. Date: Feb. 29, 2024

(54) ORTHODONTIC DEVICES AND SYSTEMS AND METHODS FOR USING SUCH DEVICES

(71) Applicant: THE BOARD OF TRUSTEES OF THE LELAND STANFORD JUNIOR UNIVERSITY, Stanford, CA (US)

(72) Inventor: HyeRan Choo, Palo Alto, CA (US)

(21) Appl. No.: 18/387,763

(22) Filed: Nov. 7, 2023

Related U.S. Application Data
(63) Continuation of application No. PCT/US22/28389, filed on May 9, 2022.
(60) Provisional application No. 63/186,161, filed on May 9, 2021.
(60) Provisional application No. 63/190,696, filed on May 19, 2021.

(51) Int. Cl. A61N 1/05; A61N 1/36

(52) U.S. Cl. CPC A61N 1/0548; A61N 1/36031; A61N 1/36078

(57) ABSTRACT

Devices and methods for treating obstructive sleep apnea, habitual mouth breathing, and/or myofunctional therapy includes a body including an anterior end, a posterior end, and lateral portions shaped for introduction into a subject's oral cavity to position the anterior end adjacent front teeth of the subject and the posterior end adjacent the pharyngeal airway of the subject with or without the posterior end adjacent the pharyngeal airway. Sensors and/or stimulators may be provided, to generate electrical current, chemical release, vibration, and/or other stimulations, e.g., to activate neuromuscular contraction, stimulate salivation and subsequent swallowing movement, and/or induce partial wakefulness of the subject and/or to record data regarding the subject's tongue movements and/or position. Optionally, an extraoral sensor device may be provided for placement around the face, nose, check, abdomen, or neck, which may be paired with the intraoral device to detect the respiratory effort related airway obstruction.

### RELATED APPLICATION DATA

The present application is a continuation of co-pending International Application No. PCT/US2022/028389, filed May 9, 2022, which claims benefit of U.S. provisional application Ser. No. 63/186,161, filed May 9, 2021, and 63/190,696, filed May 19, 2021, the entire disclosures of which are expressly incorporated by reference herein.

### TECHNICAL FIELD

The present application is related to devices and methods for treating obstructive sleep apnea (“OSA”), e.g., in neonates, infants, pediatric patients, and adults. In addition, the devices and methods herein may serve as an effective tool in objectively quantifying, training, monitoring, and/or recording the tongue movements and/or positions, e.g., related to myofunctional therapy, OSA diagnostics, and/or correction or prevention of mouth breathing habits while asleep and/or awake.

### BACKGROUND

Continuous positive airway pressure (“CPAP”) therapy is currently the first line of non-surgical treatment when a patient is diagnosed with OSA. CPAP devices provide pneumatic stenting of the upper airway and prevent the airway from collapsing during the inspiratory phase of breathing. CPAP may be very effective when the devices are properly worn. The compliance to CPAP therapy, however, is very low due to discomfort related to ill-fitting mask adaptation, high inspiratory and expiatory pressures, and bulkiness of the device. Alternatively, oral mandibular advancement devices (“MAD”) are available.

They are retained by the full upper and lower dentitions and protrude the lower jaw to enlarge the pharyngeal airway by bringing the tongue forward with the protruded lower jaw. The compliance rate with MAD is much higher although the efficacy is less than that of CPAP. MAD often results in moderate to severe side effects, such as temporomandibular joint discomfort and occlusal changes.

Hypoglossal nerve stimulation (“HNS”) surgery is effective in contracting the genioglossus muscles and superficial tongue muscles. Obviously, however, patients need to undergo major surgery under general anesthesia followed by potentially repeated surgeries in adjusting the implanted sensors and stimulators in the following years. The potential effectiveness of myofunctional therapy (“MFT”) for the tongue has been recognized in the treatment of obstructive sleep apnea. Currently, however, there is no tool available that can measure and record tongue positions inside the mouth, which has hindered scientific advancement of the treatment. Instructions for MFT for tongue exercise and treatment outcome assessments, therefore, have remained ambiguous due to the lack of quantitative outcome measures. In fact, improper tongue positioning and high arch palate caused by habitual mouth breathing during childhood have been recognized as a causal factor for OSA for children and adults. Precise quantification of tongue positions and pressure can be used to develop a powerful tool in correcting and preventing habitual mouth breathing from early childhood to adulthood, which will have a life-long positive effect in preventing or reducing OSA symptoms.

Accordingly, devices and methods for treating obstructive sleep apnea (OSA) and/or for myofunctional therapy would be useful.

### SUMMARY

The present application is directed to devices and methods for treating obstructive sleep apnea (OSA), e.g., in neonates, infants, pediatric patients, and adults. In addition, the devices and methods herein may be used for objectively quantifying, training, monitoring, and/or recording tongue movements and/or positions, e.g., related to myofunctional therapy, OSA diagnostics, and/or correction or prevention of mouth breathing habit while asleep and/or awake.

In an exemplary aspect, a device is provided that may secure or enlarge the pharyngeal airway while a patient is asleep, e.g., whenever there is a sign of upper airway obstruction, such as snoring or narrowing of the airway behind the tongue without side effects shown when using CPAP, MAD, or even the surgical hypoglossal nerve stimulation treatment. Contrary to MAD, the device does not disrupt the temporomandibular joints nor induce unfavorable occlusal changes. In addition, the device is not bulky and does not force the mouth open while wearing the device inside the mouth. Contrary to CPAP, the device may not require additional apparatus attached to the face unless the pharyngeal components of the device are omitted. The device also does not create noisy airway pressure sounds through the night.

The device may also be a useful tool in objectively quantifying the tongue positions inside the mouth while awake and/or asleep, and/or may provide valuable insights on differences how tongue position, while awake, may contribute to the treatment of tongue-based airway obstruction while asleep. Contrary to hypoglossal nerve stimulation surgery, the device does not require surgery to stimulate the hypoglossal nerve to move the tongue away from the pharyngeal airway. Since the device does not require a strong electric current to stimulate the hypoglossal nerve, nerve stimulation may be provided much more comfortably. The device may also enable large data collection, e.g., of pharyngeal airway obstruction patterns of OSA, tongue position changes while asleep, and/or correlation between healthy breathing related normative tongue positioning while awake. These data may revolutionize diagnosis, treatments, and prevention of OSA.

The devices and methods herein may be used to prevent and/or treat OSA while a patient is asleep without involving surgery by controlling the tongue via one or more of the following mechanisms. First, the devices may elevate tongue position, which subsequently may elevate the hyoid bone and enlarge the hypo- and oro-pharyngeal space. Second, the devices may let the tongue comfortably rest on pads of the device body positioned on the floor of the mouth, which may induce intrusive force on the lower molars which, in return, may induce counterclockwise rotation of the lower jaw, which may let the lower jaw naturally advance forward without artificially induced discomfort at the temporomandibular joints. Third, the elevated tongue in full contact with the palate may naturally provide the leeway between the maxillary and mandibular dentition for more physiologic comfort at the temporomandibular joint. Fourth, the devices may provide a mechanical barrier behind the base of the tongue when the tongue falls backward while asleep. The device may secure continued airway patency even though the tongue loses its tonicity completely during the rapid eye movement (“REM”) stage of sleep, during which a patient is more prone to experience complete airway obstruction. Fifth, the devices may stimulate the salivary glands to produce saliva when obstructive respiratory events occur. For example, this may trigger the natural swallowing reflex while asleep, which may naturally open the pharyngeal airway by moving the tongue away from the pharynx. The source of stimulations may be electrical, chemical, aromatic, etc. In devices where this mechanism is not utilized or there is no need for placement of sensors behind the base of the tongue, the portion of the device behind the base of the tongue may be omitted to allow for a smaller device and for greater comfort.

In an alternative example, an intraoral device may be provided that includes one or more sensors and/or stimulators on a body of the device, and the portion of the intraoral device behind the base of the tongue may be omitted. One or more extraoral sensors may be provided, e.g., which may be secured around the face, neck, chest, or abdomen of the user, e.g., at the level of nose, hyoid bone, or trachea. Optionally, the extraoral sensors may remain detached from body parts, e.g., placed in proximity to the subject. In one example, the extraoral sensor(s) may be configured to sense airway obstruction and send activation signals wirelessly to neuromuscular stimulators that are provided on the intraoral device, e.g., under the tongue to stimulate the hypoglossal nerve for genioglossus muscle and/or Vagus nerve for palatoglossus muscle. Alternatively, the intraoral device may include all necessary sensors and stimulators inside the sublingual ramps without needing extraoral sensors.

The intraoral device may include one or more sensors that are configured to sense an event of airway obstruction, e.g., using sensors that may detect changes in one or more of sound, acoustic wave, vibration, textile, contact, gravity, volume, temperature, humidity, pressure stress, movement of tissue or anatomic structure, and/or strain inside the mouth and/or outside the mouth by the pharyngeal component of the device or an extraoral device may include one or more wireless sensors, which may identify such events. The intraoral device may instantaneously trigger necessary stimulations when such events are detected to activate the major pharyngeal dilator muscles before the obstructive event occurs because the neuromuscular-, chemical-, aromatic-, acoustic-, or vibration-stimulators are located immediately under the tongue where the branches of hypoglossal nerve are distributed.

In addition, if desired, the embedded sensors around the tongue may quantify the tongue positions while asleep or while awake, which may enable precise tongue strengthening exercise prescription and/or monitoring, leading to enhanced research and treatments using myofunctional therapy for speech articulation therapy as well as for OSA treatment that is related to tongue muscle training, such as correcting habitual mouth breathing while awake. The device may not disrupt the temporomandibular joints nor induce unfavorable occlusal changes, unlike MAD. In addition, the device may secure a patent upper airway at all times without requiring CPAP masks or uncomfortable air pressure. The device may also enable large data collection on pharyngeal airway obstruction patterns of OSA, tongue position changes while asleep, and/or correlation between healthy breathing related normative tongue positioning while awake. The collected data on tongue positions and movements may unlock mysteries in oropharyngeal structure and/or may discover the etiology of functional disorders, such as sleep related breathing disorders, dysphasia, and/or speech disturbances. These data may contribute to scientific discovery that may lead to revolutionizing the diagnosis, treatments, and prevention modalities of OSA, complex dysphasia, habitual mouth breathing, and speech disorders and ultimately the overall quality of life. Additional configurations will be apparent to one of ordinary skill in the art in view of the teachings herein.

Currently disclosed herein is merely an exemplary device having components that allow for the enlargement of the pharyngeal airway to treat OSA and for the development of the objective data collection device to quantify the tongue position and tongue movements using various sensors and stimulators that are embedded in the device. The sensors may include, but are not limited to, those that detect upper airway obstruction such as sound, acoustic wave, vibration, textile, contact, gravity, volume, temperature, humidity, or pressure stress, movement of tissue or anatomic structure, and/or strain inside the mouth and/or outside the mouth. The stimulators may include, but are not limited to, devices that deliver electrical current, chemical or aroma release, vibration, and/or sound that may activate neuromuscular contraction or stimulate salivation and subsequent swallowing movement or induce partial wakefulness or any other suitable stimulation as would be apparent to one of ordinary skill in the art in view of the teachings herein.

In accordance with one example, a device is provided for treating obstructive sleep apnea that includes a body comprising an anterior portion, a posterior portion, and a pair of lateral portions extending between the anterior and posterior portions shaped for introduction into a subject's oral cavity to position the anterior portion adjacent front teeth of the subject and the posterior portion adjacent the pharyngeal airway of the subject; and one or more sensors or stimulators on the body.

In accordance with another example, a device is provided for treating obstructive sleep apnea that includes an intraoral device comprising a body shaped for introduction into a subject's oral cavity to position an anterior portion adjacent front teeth of the subject and lateral portions adjacent lower molars of the subject.

In accordance with still another example, a device is provided for treating obstructive sleep apnea that includes a body comprising an anterior portion, a posterior portion, and a pair of lateral portions extending between the anterior and posterior portions shaped for introduction into a subject's oral cavity to position the anterior portion adjacent front teeth of the subject and the posterior portion adjacent the pharyngeal airway of the subject; one or more sensors on the body one or more stimulators on the body; and a processor coupled to the one or more sensors and stimulators, the processor configured to receive signals from the one or more sensors and, when the processor identifies an event of airway obstruction based at least in part on the signals, the processor activates the one or more stimulators.

In accordance with another example, a system is provided for treating obstructive sleep apnea that includes an intraoral device comprising a body shaped for introduction into a subject's oral cavity to position an anterior portion adjacent front teeth of the subject and lateral portions adjacent lower molars of the subject, the body comprising one or more stimulators and a processor; and an extraoral device configured for placement on the subject's neck, nose, chest, abdomen or face outside the oral cavity or that remains completely detached from body parts, the extraoral device comprising one or more sensors, wherein the processor is configured to receive signals from the extraoral device and, when the processor identifies an event of airway obstruction, the processor activates the one or more stimulators.

In accordance with yet another example, a system is provided for treating obstructive sleep apnea that includes an intraoral device comprising a body shaped for introduction into a subject's oral cavity to position an anterior portion adjacent front teeth of the subject and lateral portions adjacent lower molars of the subject, the body comprising one or more sensors and a intraoral communications interface; and an extraoral electronic device comprising a processor, an extraoral communications interface, and an output device, wherein the processor is configured to receive signals from the one or more sensors of the intraoral device via the intraoral and extraoral communications interface and process the signals to identify when the subject's tongue contacts the one or more sensors, the processor configured to present information regarding the contact on the output device to provide feedback to the subject regarding the contact.

In accordance with another example, a method is provided for treating obstructive sleep apnea that includes introducing an intraoral device into a subject's oral cavity such that an anterior portion of the device is positioned adjacent front teeth of the subject, a posterior portion of the device is positioned adjacent the pharyngeal airway of the subject; and activating the device such that one or more sensors monitor one or more parameters of the subject during sleep and, when one or more conditions are met, the device delivers stimulation to the tongue to prevent or treat sleep apnea.

In accordance with still another example, a method is provided for treating obstructive sleep apnea that includes introducing an intraoral device into a subject's oral cavity such that an anterior portion of the device is positioned adjacent front teeth of the subject and lateral portions of the device are positioned adjacent lower molars of the subject, the device carrying one or more stimulators; and activating the device such that one or more sensors monitor one or more parameters of the subject during sleep and, when one or more conditions are met, the device delivers stimulation to the tongue via the one or more stimulators to prevent or treat sleep apnea.

In accordance with yet another example, a method is provided for inserting a device into a subject's oral cavity without triggering gagging reflex that includes providing an intraoral device comprising a body shaped for introduction into the subject's oral cavity to position an anterior portion adjacent front teeth of the subject and lateral portions adjacent lower molars of the subject; before inserting the device, extending the subject's tongue out of the subject's mouth; passing the subject's tongue through the space between the lateral portions while the tongue is still extending with the mouth open; introducing the tongue and the device together into the inside of the mouth; and closing the mouth and immediately initiating breathing through the nose.

In accordance with still another example, a method is provided for making a device for treating obstructive sleep apnea, the method including forming an intraoral device comprising a body shaped for introduction into a subject's oral cavity to position an anterior portion adjacent front teeth of the subject and lateral portions adjacent lower molars of the subject, and including one or more stimulators and/or sensors.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A is a superior view of an example of an intraoral device.

FIGS. 1B and 1C are frontal and posterior views, respectively, of the intraoral device of FIG. 1B.

FIG. 2 shows an alternative intraoral device without the posterior components of the device shown in FIGS. 1A-1C.

FIG. 3 is a schematic showing exemplary components of a system including an intraoral device and external components, e.g., one or more wearable sensors, a charger, and a remote control.

FIGS. 4A-4C show various views of the intraoral device of FIGS. 1A-1C placed on a mandibular dentition model positioned relative to a subject's teeth.

FIG. 5 shows a lateral view of the intraoral device of FIGS. 1A and 1B positioned relative to a subject's tongue.

FIG. 6 shows the intraoral device of FIG. 2 positioned within a subject's oral cavity and an external wearable device carrying one or more sensors.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Generally, the intraoral devices herein are removable orthodontic devices for treating obstructive sleep apnea (“OSA”) and/or other conditions, e.g., for myofunctional therapy, in neonates, infants, pediatric patients, and adults. In addition, the devices and methods herein may be used for objectively quantifying, training, monitoring, and/or recording the tongue movements and/or positions, e.g., related to myofunctional therapy, OSA diagnostics, and/or correction or prevention of mouth breathing habit while asleep and/or awake. The devices may be operable for use by itself or may be included with a system, e.g., including one or more wearable or separate sensors and stimulators, a remote control or other electronic device, and the like, as described further elsewhere herein.

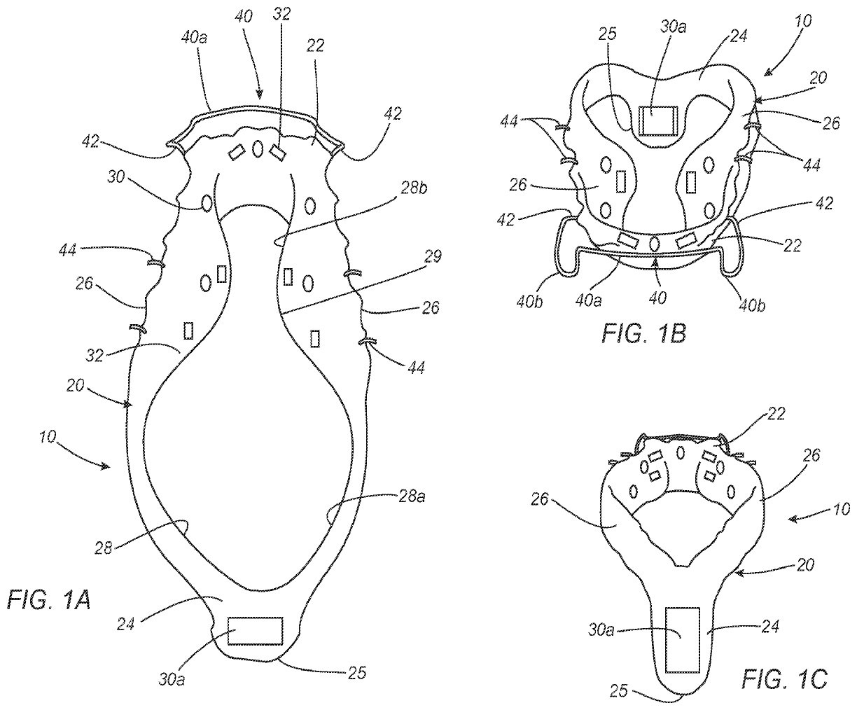

Turning to the drawings, FIGS. 1A-1C show an example of an intraoral device 10 including a body 20 sized and/or shaped for introduction into a subject's oral cavity carrying one or more sensors 30 and/or stimulators 32. Generally, the body 20 includes an anterior end or portion 22, a posterior end or portion 24, and a pair of lateral portions 26 extending between the anterior and posterior portions 22, 24 sized and/or shaped to position the anterior portion 22 adjacent front teeth of the subject and the posterior portion 24 adjacent the pharyngeal airway of the subject.

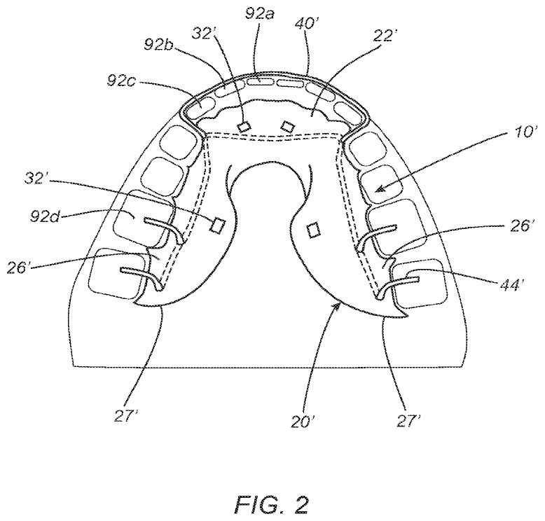

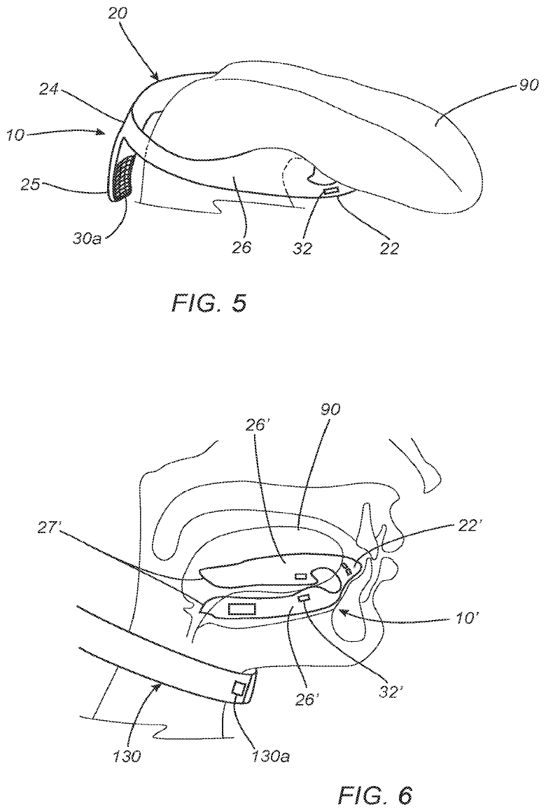

For example, as shown, the anterior and lateral portions 22, 26 may be shaped to provide a sublingual platform surrounding an open space 28 configured to receive the base of a tongue 90 of the subject (e.g., as shown in FIG. 5), and the posterior portion 24 may be shaped to include sublingual faucial wraps, a retroglossal pad, and/or a pharyngeal tail configured to extend partially into the subject's pharyngeal airway during use. Alternatively, as shown in FIG. 2, the posterior portion may be omitted to provide a device 10′ that includes only the anterior portion 22′ and lateral portions 26.′ In this alternative, one or more extraoral sensors may be provided on a wearable or other external device 130, e.g., as shown in FIG. 6 and described elsewhere herein.

The body 20 may be shaped such that the anterior portion 22 may be positioned sublingually when the tongue 90 is received through the open space 28, with the lateral portions 26 shaped to contact the subject's tongue 90, e.g., to place the sensor(s) 30 and/or stimulator(s) 32 in contact with the tongue 90, e.g., as shown in FIG. 5 and described further elsewhere herein.

In one example, the sublingual platform may define a key hole shaped open space 28, e.g., including a larger posterior region 28a and a narrower anterior region 28b, that may provide room for the lingual frenum and allow for the anterior projection and lateral movements of the tongue tip without limitation. For example, the narrow anterior region 28b of the open space 28 may be sufficiently large to provide room for the anatomy of the lingual frenum at rest and during the anterior projection of the tongue tip, while guiding the middle third and posterior third of the tongue's total length to remain relatively in the median plane. As a reference, the lingual frenum is located in the median plane on the ventral (inferior) surface of the tongue. The superior or upper surface 29 of the sublingual platform may have a gentle concave slope down towards the lingual frenum to accommodate the natural convexity of the ventral (inferior) surface of the tongue.

In various examples, the body 20 of the device 10 (or any of the other devices herein) may be made from acrylic or other plastic, metal, composite, or other biocompatible materials, e.g., by one or more of molding, casting, 3D printing, machining, and the like, with the sensors 30 and stimulators 32 embedded in or attached to one or more surfaces of the body 20. The body 20 may be substantially rigid or may be malleable such that the shape of the body 20 may be modified, if desired.

Optionally, one or more retention elements may be provided on the body 20 to secure and/or stabilize the device 10 within the subject's oral cavity. For example, as best seen in FIGS. 1A and 1B, a labial bow element 40 may be secured at the anterior end 22 that is shaped to surround or otherwise engage one or more of the subject's teeth. For example, the labial bow element 40 may include a horizontally curved region 40a shaped to contact the mandibular anterior teeth, e.g., the central and lateral incisors 92a, 92b with or without the canines 92c, e.g., including lateral regions 40b shaped to avoid contact with the canines 92c. In one example, a rigid wire, e.g., formed from stainless steel or other metal, plastic, composite, and/or other biocompatible material, may include opposite ends 42 that are secured to the body 20, e.g., by one or more of gluing, screwing or otherwise inserting the ends 42 into holes or recesses in the body 20, molding the ends 42 into the body 20, and the like. The wire may be substantially rigid or may be malleable, e.g., so that the subject can adjust the shape of the element 40, if desired. Optionally, the occlusal surface of the anterior teeth alone or the entire mandibular dentition may be covered by acrylic or plastic material to provide disclusion from the maxillary dentition or as part of a retention component for the device. Generally, the retention element 40 may ensure that the body 20 of the device 10 remains on the lingual surface of the teeth 92 under the tongue 90 and the sublingual wraps and retroglossal pad on the posterior portion 24 extend from the sublingual region of the body 20 to allow for free movement of the palatoglossal muscle and/or to avoid triggering the gagging reflex while asleep.

In addition or alternatively, the lateral portions 26 may include one or more posts or other supports, e.g., occlusal stops 44, configured to contact lower molars 92d, e.g., the mandibular permanent molars on either side of the subject's oral cavity. In cases where mandibular permanent molars are not available, the stops 44 may be located on the body 20 such that they may be placed on the mandibular bicuspids. The occlusal stops 44 may be separate components permanently attached to the body 20, e.g., formed from the same or different material than the body 20 or labial bow element 40, or may be integrally formed, e.g., molded, cast, machined, and the like, with the body 20 from the same material. The occlusal stops 44 may further stabilize the device 10 within the subject's oral cavity in addition to the labial bow element 40. For example, the stops 44 may provide vertical support for the device 10 to prevent the body 20 from falling to the floor of the mouth when the tongue 90 is rested on the sublingual acrylic platforms. Alternatively, as shown in FIG. 2, the occlusal stops 44′ may be formed from one or more wires embedded or otherwise secured to the body 20′ that extend from one lateral portion 26′ through the anterior portion 22′ to the other lateral portion 26.′ Optionally, the labial bow element 40′ may be formed from the same wire(s) or may be coupled to the wire(s), as shown, such that the supports are a singular structure that may be embedded within the acrylic or other material of the body 20′ during manufacturing. Such a unitary support structure may be provided in the body 20 of device 10 or any other devices herein.

Optionally, the body 20 (or any of the other devices herein) may include one or more mechanical markings, such as holes, recesses, or textured regions having a desired roughness (not shown) at desired locations on the surface of the body 20, e.g., to provide tactile identification of the locations and/or guide certain positioning or movements of the subject's tongue. These mechanical marking may be located on the body 20 in conjunction with the sensors 30. For example, the mechanical markings may be located immediately adjacent sensors that may be positioned under the subject's tongue, e.g., to help subjects visualize their tongue movement and positioning habit, e.g., to establish more favorable airway tone and anatomy while they are awake.

The sensors 30 may be arranged at desired locations on the body 20 to detect one or more of sound, acoustic wave, vibration, textile, contact, gravity, volume, temperature, humidity, pressure stress, movement of tissue or anatomic structure, and/or strain inside the mouth and/or outside the mouth, as described further elsewhere herein. The stimulators 32 may include one or more neuromuscular, chemical, aromatic, vibrational mechanical, and/or electrical stimulators, e.g., configured to generate one or more of electrical current, chemical releases, aromas, vibrations, and/or sounds, e.g., to activate neuromuscular contraction, stimulate salivation and subsequent swallowing movement, and/or induce partial wakefulness of the subject, also as described further elsewhere herein.

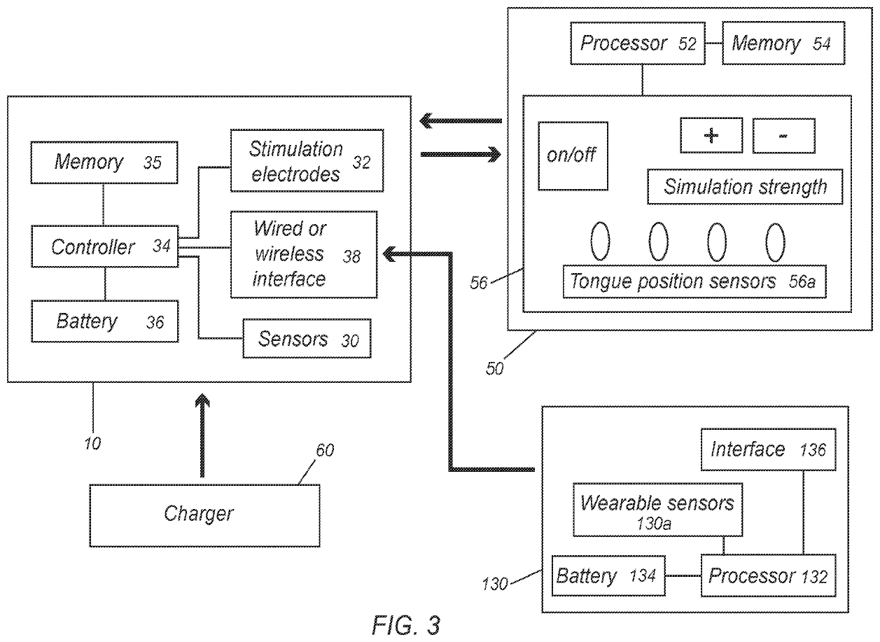

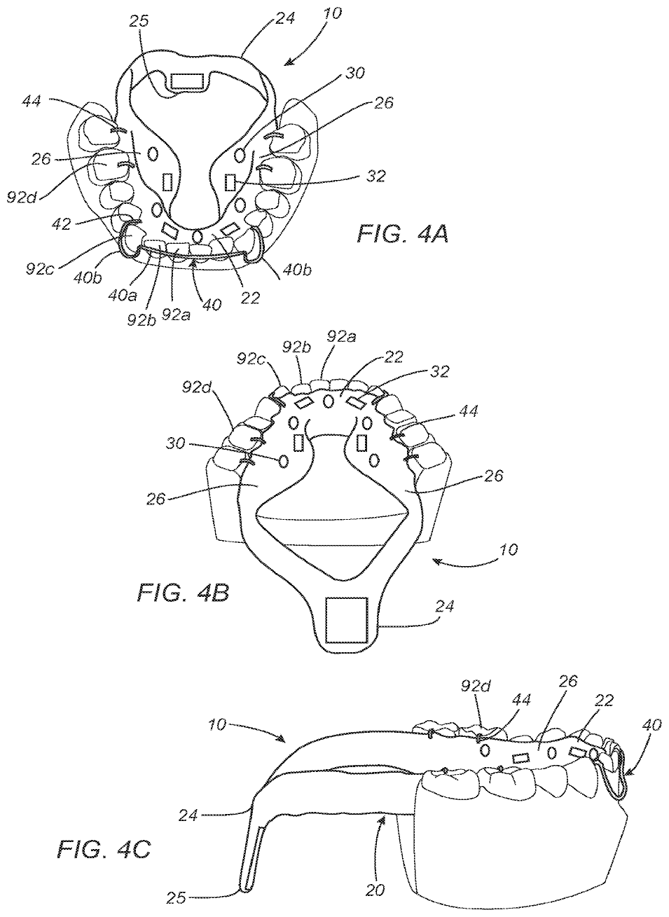

The sensors 30 and/or stimulators 32 may be coupled to and/or controlled by one or more processors or controllers, e.g., which may be configured to provide an immediate feedback system to the stimulators, e.g., for genioglossus or palatoglossus muscles. For example, as shown in FIG. 3, the device 10 may include one or more electrical components for operating the sensors 30 and/or stimulators 32, e.g., a processor or controller 34, a battery or other power source 36, and a communications interface 38, e.g., a wireless transmitter and/or receiver for communicating with one or more external devices, as described further elsewhere herein.

In one example, a set of sensors 30 and stimulators 32 may be located on the body on the sublingual platform around the key hole shape opening 28. For example, as best seen in the FIG. 1A, a plurality of stimulators 32 (represented by rectangular shapes, although the actual stimulators may have other shapes) may be located on the anterior and/or lateral portions 22, 26 to provide electrical current to activate the branches of hypoglossal nerves that distribute within the genioglossus muscles and intrinsic muscles of the tongue 90, and/or the branch of the Vagus nerve that distributes within the palatoglossus muscle. The stimulators 32 may be located on the superior or inferior surface of the sublingual acrylic platform and/or inside the platform.

In addition, a plurality of sensors 30 (represented by oval shapes, although the actual sensors may have other shapes) may be strategically located on the body 20 to capture one or more parameters, e.g., pressure change or tactile changes when the tongue 90 is in contact or out of contact with the sensor(s) 30 and/or other parameters described herein. For example, a pressure sensor may be placed on the superior surface of the sublingual platform in the mandibular anterior region. The subject may be instructed to do a specific tongue exercise while keeping the tongue tip on the sensor and/or in mechanical markings, such as holes in the body 20, with a specific amount of pressure. The pressure level and specific positioning of the tongue may be able to trigger the sensor 30 and processor 34 may record the associated data. The processor 34 may receive and process signals from the sensor(s) 30 and provide real-time feedback to the subject whether the tongue is at a correct or therapeutic position with a correct amount of pressure. In addition or alternatively, as shown in FIG. 3, the processor 34 may communicate the signals and/or data via the interface 38 to a remote electronic device 50, e.g., a monitor, remote control, or other external controller. The external device 50 may include one or more processors 52, memory 54, and/or output devices 56 to provide an indication to the subject when their tongue is properly positioned relative to the sensor(s) 30 and, consequently, within the subject's mouth. For example, the output device may include one or more lights, e.g., a green, red, yellow lighting system 56a, that may indicate proper or improper contact. In addition or alternatively, the external device 50 may include a display (not shown) that may provide additional information to the subject. In one example, the external device 50 may be a mobile phone, tablet, laptop, or other electronic device that may include an application that may provide more precise instructions, communications, monitoring, and/or data recording for the subject, e.g., for monitoring and/or guiding tongue exercises or swallowing exercises, e.g., as part of OSA prevention, myofunctional therapy, correction of dysphasia, habitual mouth breathing, or speech articulation treatment.

Optionally, the sensors 30 may be located at specific locations on the body 20, e.g., on the anterior portion 22 or different sensors may be provided throughout the body 20. For example, sensors 30 may be located on the body 20 such that the processor 34 may record information about the subject's tongue position and/or movement, which may be analyzed by the processor 34 or the external device 50, and/or may be communicated to a health care provider or other location to provide information on tongue positions when the subject is asleep or awake, which may provide diagnostic information for sleep apnea, dysphagia, speech impairments, and/or other conditions.

The electronic components of the device 10 may be embedded within and/or otherwise mounted on the body 20, e.g., to provide power to and/or control operation of the one or more sensors 30 and/or stimulators 32. The components may be provided at any desired location on the body 20, e.g., within the sublingual pad or at multiple spaced-apart locations and coupled by one or more leads (not shown). For example, a rechargeable battery 36 may be provided that may be coupled to the sensor(s) 30 and/or stimulator(s) 32 via the processor 34, which may analyze data from the sensor(s) 30 and activate the stimulator(s) 32 when appropriate, as described elsewhere herein. Optionally, the device 10 may include memory 35, e.g., coupled to the processor 34 for storing instructions and/or data from the sensor(s) 30, and/or the communications interface 38 for receiving instructions, updates, and/or transmitting data or other information. In one example, the communications interface 38 may include a wireless transceiver that may communicate with the external device 50 and/or other devices via Bluetooth or other RF protocols.

Thus, the electronics of the device 10 may be self-contained within the body 20, while allowing wireless communications and/or inductive or other charging of the battery 35. Alternatively, a connector (not shown) may be provided on the body 20 that is electrically coupled to one or more of the internal components such that, when the device 10 is not in use, an external device 60, such as that shown in FIG. 3, e.g., a charger, dedicated controller, and/or portable electronic device including appropriate software, may be connected to the device 10 to charge the battery 35, provide instructions and/or updates, and/or receive data. The connector may be formed from biocompatible material or a cover or other sealing member (not shown) may be provided that may prevent exposure of the connector during use. In a further alternative, one or more wires or cables may extend from the body 20 that are coupled to the internal components, which may be connected to an external device before/after use or even during use, if desired.

In one example, best seen in FIGS. 1A and 5, as the lateral portions 26 of the device, i.e., including right and left sublingual platforms, extends toward the posterior portion 24 (configured to be positioned at the back of the tongue 90 inside the subject's throat), the lateral portions 26 may become narrower and thinner, e.g., looking like a wrap or band around the back of the tongue 90. The posterior portion 24 may include right and left ventrolateral wraps that surround the back of the tongue 90, e.g., without interfering substantially with movement of the two faucial pillars, and converge to the median plane behind the tongue 90. The position of the ventrolateral wraps is not necessarily limited to the ventrolateral surface of the tongue 90, and may pass the two faucial pillars through under the tongue 90, sides of the tongue 90, and/or the dorsum of the tongue 90. When the right and left ventrolateral wraps proximate behind the back of the tongue base (e.g., the posterior third of the tongue 90), they may completely merge to form one retroglossal pad or tail 25, e.g., having a curved shape behind the tongue 90. Optionally, the tail of the posterior portion 24 may include an additional sensor 30a, e.g., as shown in FIGS. 1A-1C, which may be any of the sensors described elsewhere herein.

Turning to FIG. 2, when the posterior portion is omitted, i.e., such that the body 20′ includes lateral portions 26′ that terminate at posterior ends 27′, one or more extraoral sensors may be provided. For example, as shown in FIG. 6, an extraoral device 130 may be provided that includes one or more sensors 130a carried on a structure configured to be secured to the subject, e.g., around their neck, as shown, or to their chest, abdomen, nose, and/or other location(s) (not shown). In one example, the structure may include one or more straps, adhesive pads, and the like (not shown) that may be secured around the subject's body or removably secured to their skin, to which the sensor(s) 130a may be permanently attached. Thus, the device 130 may be easily secured to the subject before use and removed when not in use.

The extraoral device 130 may replace the sensors on the body or may be used in addition to such sensors. In one example, the extraoral device 130 may include a sensor 130a configured to detect respiratory related breathing efforts of the subject, e.g., resulting from an airway obstruction.

As shown in FIG. 3, the extraoral device 130 may include one or more electrical components for its operation, e.g., a processor 132, a battery or other power source 134, and/or a communication interface 136. For example, the processor 132 may receive signals from the sensor(s) 130a and transmit them to the processor 34 on the body 20 and/or to the external device 50, which may then process the signals or data, e.g., to activate stimulators 132 on the device 10.′ In addition or alternatively, one or more extraoral sensors may be provided that remain completely detached from body parts of the subject, such as wireless sound sensors that may be put on a night stand or elsewhere near the subject (not shown).

Returning to FIGS. 1A-1C, optionally, at least a portion of the body 20 may be adjustable or movable. For example, the sublingual wraps and/or retroglossal pad of the posterior portion 24 may be movable relative to the lateral portions 26 of the body 20. For example, the material may be malleable such that these portions may be shaped based on an individual subject's oral anatomy before first use, or the material may be heated and softened to allow adjustments and then cooled again to maintain the adjusted shape. Alternatively, one or more hinges, screw mechanisms, and/or other components (not shown) may be provided, e.g., on the sublingual wraps and/or retroglossal pad 25 to adjust the device 10, e.g., adjust a distance between the anterior and posterior ends 22, 24 of the device to provide desired support of the posterior region of the subject's tongue during use. The retroglossal pad 25 may include one or more retroglossal sensors 30a. In various examples, the retroglossal sensor 30a may be configured to detect, but not limited to, changes of sound, acoustic wave, vibration, air pressure, airflow velocity, contact stress, textile, contact, gravity, volume, temperature, humidity, pressure stress, movement of anatomic structures, strain, and/or deformation of the retroglossal pad itself.

In another alternative, the retroglossal pad and one or more sensors thereon may remain separated behind the tongue as an extension of the sublingual wrap from each side of the tongue. For example, the device may include two retroglossal pads extending from respective lateral portions of the device, and retroglossal sensors may be provided on each extension, e.g., separated in the median plane. The two retroglossal pads may be remain separate or may be coupled together by one or more connectors (not shown).

Ideally, the retroglossal pad or tail 25 may not be in contact with the posterior third of the tongue when a patient is awake. When the patient is asleep and the tongue starts to prolapse, narrowing the airway and vibrating the soft palatal tissues, then the intraoral or extraoral sensors 30 may detect these signs of reduced or interrupted airflow and immediately trigger forward tongue movement, e.g., by activating one or more of the stimulators, e.g., those located at the sublingual pads on the lateral portions 26 around the key hole region 28b of the opening 28. In one example, the tongue movements necessary to open up the airway may be triggered by direct neuromuscular stimulation to the hypoglossal nerve or parasympathetic neurosensory stimulation that may trigger excessive saliva production and its subsequent natural swallow movements of the tongue. Natural saliva swallowing may trigger the back of the tongue to move upward to make a full contact with the soft palate, away from the obstruction of the airway. Alternatively, other forms of stimulators may be used, e.g., electrical, chemical, aromatic, and the like, to stimulate the salivary glands to produce saliva when obstructive respiratory events occur. This may trigger the natural swallowing reflex while asleep, which may naturally open the pharyngeal airway by moving the tongue away from the pharynx.

With continued reference to FIG. 1A, the open space 28 between the sublingual pads may extend from the anterior floor of the mouth to the area behind the tongue of the subject. In the example shown, when viewed from above, the opening may have a bowling pin shape although other shapes may be provided. As mentioned above, the smaller, anterior region 28b (the key hole shaped space that is located at the anterior of the mouth) is intended to provide full freedom for tongue tip movements and giving relatively limited freedom for lateral movements of the tongue body. The larger, posterior region 28a of the opening 28 is intended to receive the tongue base and to allow vertical movement of the tongue whenever the tongue needs to move upward to meet the soft palate. Generally, the body 20 should be configured such that the tongue may not have limitation of its movement with regards to the upward and forward moment.

Returning to FIGS. 1A-1C and 5, an exemplary and novel method is now described for how the device 10 may be inserted under and toward the back of the tongue 90 without triggering gagging reflex. Before inserting the device 10, the subject is instructed to stick out the tongue 90, e.g., as far forward as possible. Then, the device 10 is put on by passing the tongue 90 through the open space 28 while the tongue 90 is still protruded with the mouth open. Next, the subject may be instructed to bring the tongue 90 and the device 10 together into the inside of the mouth and close the mouth and immediately initiate breathing through the nose. This may ensure that the tongue 90 does not recognize the device 10 as a hazardous foreign body that may slide down on the dorsum of the tongue 90 toward the throat by accident, and, therefore, ensure that gagging reflex is not triggered.

Alternatively, with respect to the device 10′ shown in FIGS. 2 and 6 (where the faucial wraps, retroglossal pad, and retroglossal sensor are omitted), an extraoral sensor device 130 may be provided that is configured to be secured around, but not limited to, or otherwise to the face, neck, chest, or abdomen of the user, which may be paired with the intraoral device 10′ to detect the respiratory effort related airway obstruction, e.g., using Bluetooth or other wireless communications. In addition or alternatively, as described elsewhere herein, extraoral sensors may be provided that remain completely detached from the body of the subject, such as wireless sound sensors that may be placed on a night stand in the subject's bedroom or elsewhere in close proximity to the subject.

With this device 10′, the anterior, right, and left sublingual platform of the anterior portions 26′ may function to provide gentle mechanical lifting of the tongue body, e.g., helping to lift the hyoid bone forward and upward while enabling the dorsum of the middle third of the tongue to remain in full contact with the posterior palate.

In young children, full constant contact and gentle continuous suction created by the tongue and the palate while swallowing saliva or during nasal breathing stimulates to widen the palate and nasal cavity, which naturally reduces resistance of air flow through the nose. This is a very important phenomenon in promoting healthy normal upper airway development for growing children. With insufficient growth and development of the upper airway anatomy resulted from abnormal mouth breathing habit and chronic habitual abnormal tongue positioning, the risk of developing OSA in later in life increases dramatically due to aging-related muscular hypotonicity and delayed response to respiratory motor output.

The device 10 of FIGS. 1A-1C may be used to provide anterosuperior repositioning of the hyoid bone to enlarge the pharyngeal airway. In addition, the right and left sublingual wrap and retroglossal pad on the posterior portion 24 may function to provide a mechanical barrier against the tongue base to secure the airway behind the tongue when the muscular tone of the tongue decreases and starts to prolapse while the subject is asleep. The vertical length and angulation of the retroglossal pad may vary depending on the subject's particular anatomy of the throat, soft palate, uvula, and the obstruction site of the airway, etc., although the bottom end of the retroglossal pad may certainly stay above the fully elevated epiglottis.

It is to note that the entire body of the device 10 (or any of the devices herein) may be made of the same material (e.g., plastic, acrylic, metal or hybrid material, etc.) or a combination of different materials that are optimized for functions of each part of the device 10. In addition, various mechanisms that may adjust the shape, size, or volume of the sublingual wraps and retroglossal pad of the posterior portion 24 may be incorporated in the body 20 of the device 10. Optionally, the device 10 may include a mobilizing mechanism, e.g., for the retroglossal pad and/or sublingual wraps in cases where more active mechanical separation of the tongue away from the posterior pharyngeal wall is desired.

In an exemplary method, the device 10 may be fully custom-fabricated and

adjusted based on one or more intraoral scanning, intrapharyngeal scanning, oral impressions, cone beam computerized tomography (CBCT), magnetic resonance imaging (MRI), and/or clinical intraoral adjustments. When the customization process is completed and the user feels comfortable with wearing the finalized device, a special service of 3D scanning and banking of the uniquely custom-finalized device may be offered so that a duplicate may be easily produced using 3D printing technology, e.g., if the finalized device is damaged or lost. However, this concept may also be expanded to include non-customized design and fabrication of the device. Optionally, the device may be produced using Computer Aided Design and Computer Aided Manufacturing (CAD CAM) and three-dimensional printing technology.

Optionally, in addition to an intraoral device that is introduced in the lower jaw here, another intraoral device (not shown), e.g., an acrylic or plastic device intended to cover the entire surface of the palate and the upper teeth may also be provided that includes one or more pressure, tactile, or other sensors. This upper intraoral device may be paired up with the lower jaw device, which may together provide more comprehensive recording of data related to tongue movement and positions using the devices. It will be appreciated that other exemplary uses of the reconstitution device may be used as would be apparent to one of ordinary skill in the art in view of the teachings herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for treating obstructive sleep apnea, comprising:
a body comprising an anterior portion, a posterior portion, and a pair of lateral portions extending between the anterior and posterior portions shaped for introduction into a subject's oral cavity to position the anterior portion adjacent front teeth of the subject and the posterior portion adjacent the pharyngeal airway of the subject; and
one or more sensors or stimulators on the body.

2-4. (canceled)

5. The device of claim 1, further comprising a retention component on the anterior portion configured to engage one or more of the front teeth to prevent migration of the body.

6. The device of claim 1, further comprising one or more stops on the lateral portions for contacting one or more teeth to provide vertical support for the body.

7. The device of claim 1, wherein the lateral portions comprise sublingual platforms configured to support a tongue of the subject and where the lateral portions define an open space between them to accommodate the tongue.

8. The device of claim 1, wherein the one or more sensors or stimulators comprise one or more stimulators on the anterior portion and/or lateral portions.

9. The device of claim 8, wherein the one or more stimulators are configured to generate, but not limited to, one or more of electrical current, chemical, aroma, release, vibration, or sound.

10. The device of claim 8, wherein the one or more stimulators are configured to activate neuromuscular contraction, stimulate salivation and subsequent swallowing movement, or induce partial wakefulness of the subject.

11. The device of claim 1, wherein the one or more sensors or stimulators comprise one or more pressure sensors on the anterior portion and/or lateral portions.

12. The device of claim 1, wherein the posterior portion comprises a retroglossal pad or tail shaped to support a posterior region of the subject's tongue.

13. The device of claim 1, wherein the one or more sensors or stimulators comprise a sensor on the posterior portion configured to detect changes, but not limited to, in one or more of sound, acoustic wave, vibration, air pressure, airflow velocity, contact stress, textile, contact, gravity, volume, temperature, humidity, pressure stress, movement of anatomic structures, strain, and/or deformation.

14. The device of claim 1, wherein the one or more sensors or stimulators comprise one or more stimulators on the anterior portion and/or lateral portions, and wherein the device further comprises:
an extraoral device configured for placement on the subject's neck, nose, chest, abdomen, or face outside the oral cavity or that remains completely detached from body parts, the extraoral device comprising one or more sensors.

15. The device of claim 14, wherein the extraoral device comprises one or more wireless sound sensors configured to be placed in proximity to the subject, such as on a night stand next to the subject's bed.

16. The device of claim 14, wherein the extraoral device is configured to communicate wirelessly with the device introduced into the subject's oral cavity.

17. The device of claim 16, further comprising a processor on the body coupled to the one or more stimulators, the processor configured to receive signals from the extraoral device and, when the processor identifies an event of airway obstruction, the processor activates the one or more stimulators.

18. A system for treating obstructive sleep apnea, comprising:
an intraoral device comprising a body shaped for introduction into a subject's oral cavity to position an anterior portion adjacent front teeth of the subject and lateral portions adjacent lower molars of the subject, the body comprising one or more stimulators and a processor; and
an extraoral device configured for placement on the subject's neck, nose, chest, abdomen or face outside the oral cavity or that remains completely detached from body parts, the extraoral device comprising one or more sensors,
wherein the processor is configured to receive signals from the extraoral device and, when the processor identifies an event of airway obstruction, the processor activates the one or more stimulators.

19. The system of claim 18, wherein the extraoral device comprises one or more wireless sound sensors configured to be placed in proximity to the subject, such as on a night stand next to the subject's bed.

20. The system of claim 18, wherein each of the intraoral and extraoral devices comprises a wireless communication interface for communicating signals from the one or more sensors to the processor.

21. The system of claim 18, wherein the one or more stimulators are configured to generate one or more of electrical current, chemical release, or vibration.

22-32. (canceled)

33. A method for treating obstructive sleep apnea, comprising:
introducing an intraoral device into a subject's oral cavity such that an anterior portion of the device is positioned adjacent front teeth of the subject, a posterior portion of the device is positioned adjacent the pharyngeal airway of the subject; and
activating the device such that one or more sensors monitor one or more parameters of the subject during sleep and, when one or more conditions are met, the device delivers stimulation to the tongue to prevent or treat sleep apnea.

34-47. (canceled)
